(19) United States
(12) Patent Application Publication
(10) Pub. No.: US 2023/0270679 A1
(43) Pub. Date: Aug. 31, 2023

(54) METHOD FOR THE PRODUCTION OF BIOCOMPATIBLE NANOMATERIALS WITH SELECTIVE RECOGNITION CAPABILITIES AND USES THEREOF

(71) Applicants: Devid MANIGLIO, (US); Alessandra Maria BOSSI, (US)

(72) Inventors: Devid MANIGLIO, (US); Alessandra Maria BOSSI, (US)

(21) Appl. No.: 18/248,649

(22) PCT Filed: Oct. 15, 2021
(86) PCT No.: PCT/IB2021/059497

§ 371 Date: Apr. 11, 2023

(30) Foreign Application Priority Data
Oct. 15, 2020 (IT) 102020000024289

(51) Int. Cl. A61K 9/16; G01N 33/58

(52) U.S. Cl. CPC A61K 9/1658; G01N 33/582

(57) ABSTRACT

A molecularly imprinted polymer in the form of nanoparticles, a method of preparation and uses thereof. Polymeric nanoparticles have recognition sites of at least one target molecule and are obtained by crosslinking of at least one polymer that is functionalized at least with polymerizable double bonds, in a liquid and in the presence of at least one target molecule as template molecule. Polymeric nanoparticles can be used for many applications, such as selective recognition of analytes, in vivo and in vitro targeting, labelling of biological molecules or in the preparation of molecular sensors.

The present invention relates to the preparation of biocompatible polymeric nanomaterials and their uses in the fields of selective recognition of specific target molecules and/or interaction with cells, bacteria or viruses. Such nanomaterials are prepared starting from at least one natural polymer, which is modified by the covalent addition of reactive chemical functional groups, solvated in aqueous solvents and stabilised by polymerization. Specific binding sites for molecule recognition are formed on biocompatible nanomaterials by molecular imprinting processes.

As is well known, the molecular imprinting technique allows selective binding sites to be formed in polymeric materials through the formation of covalent, or even non-covalent, interactions between a “template molecule” (or templating agent) and a selection of functional monomers, i.e. a set of building elements of the molecularly imprinted polymer (EP0602154A1; Arshady R. and Mosbach K. Makromol. Chem. 1981, 182:687; Andersson L. et al., Tetrahedron Lett. 1984, 25:5211; Wulff G. and Sarhan A. Angew Chem Int Ed 1972, 11:341; EP1144007A; WO0041723A1; EP0102985A1; EP0602154A1).

The concept of molecular imprinting involves the polymerization of functional monomers, i.e. monomers with at least one polymerizable functional group, in the presence of a template molecule, which may be the whole or part of the molecular target, or a chimera thereof. At the end of the polymerization process, the removal of the template molecule from the formed material is carried out, making accessible molecular cavities having a shape complementary to the template itself and in which the binding functional groups are immobilized in a spatial configuration that is complementary to the template molecule. This results in a high and specific selectivity of the polymeric material produced towards the template molecule, which is the molecular target of interest or part of it or a chimera thereof. A summary of the technique is provided by Ekberg B. and Mosbach K., in Trends Biotechnol. 1989, 7:92.

Modifications to the molecular imprinting process allow the preparation of selective binding materials of nanometric size and therefore similar to antibodies, sometimes also called artificial antibodies (CN102114416A; WO2013041861A2).

Molecularly imprinted polymers, when prepared in nanometre dimensions by one of the methods known in the art (Hoshino Y. et al. J. Am. Chem. Soc. 2008, 130:15242; Perez-Moral N. and Mayes A.G. Langmuir 2004, 20:3775; Poma A. et al. Adv. Funct. Mater. 2013, 23:2821; Ambrosini S. et al. Chem. Comm. 2013, 49:6746), show high affinity and selectivity towards the template molecule, or part of it or a chimera thereof, and, in analogy to natural antibodies, exert the function of selectively binding a target molecule, even when it is located in biological contexts, in vitro and in vivo, including the bond of said molecule when it is intracellular or present on cell membranes (Piletsky S. et al. Trends Biotechnol 2020, 38:368; Haupt K. et al. Chem. Rev. 2020, 120:9554).

In the state of the art, molecularly imprinted polymers in the form of nanoparticles are mostly obtained through synthesis methods in which the set of building elements used to form the polymer consists of one or more small monomers, mainly (meth)acrylates and (meth)acrylamides, as described for example in US 2009/0311324 A1. However, polymers prepared starting from these monomers are not degradable materials and therefore pose a potential risk of accumulation both in organisms, for example when used in vivo for diagnostic and therapeutic purposes, and in the environment.

For this reason, more recently, for preparing molecularly imprinted polymers in the form of nanoparticles, it has been proposed to use sets of building elements based on mixtures of the above-mentioned conventional monomers (i.e. (meth)acrylamides and (meth)acrylates) with naturally occurring and degradable building elements, such as polysaccharides like chitosan.

However, polysaccharides are building elements characterized by a high repetitiveness of structure and containing only OH groups as functional groups capable of interacting with the target molecule. The use of polysaccharides as building elements therefore has the disadvantage of limiting the affinity and selectivity of the molecularly imprinted polymers that can be obtained, as well as the type of template molecules that can be used for their preparation and thus the target molecules that can be recognized. Furthermore, polysaccharides exhibit high wettability, which in preparation methods of the prior art is often modulated by the addition of synthetic hydrophobic molecules or by forming synthetic head-tail constructs. In addition, polysaccharides are often associated with the development of inflammatory states in in vivo applications, thus limiting their uses.

In the state of the art, it is also known to use molecularly imprinted polymers prepared using sets of building elements containing protein building elements, such as amino acids, peptides, polypeptides, proteins and glycoproteins mixed with the above-mentioned conventional monomers (i.e. (meth)acrylamides and (meth)acrylates). Such polymers are described, for example, in US 2003/153001 A1 and US 2004/058006 A1. In these polymers, protein building elements are used to introduce specific binding functional groups into the cavities of the recognition sites in order to improve the binding capacity and selectivity of the imprinted polymer towards the target molecule, while the structure of the polymer and the recognition cavities consists mostly of conventionally synthesized monomers such as (meth)acrylamides and (meth) acrylates.

Also known in the state of the art is the ‘bio-imprinting’ technique (Staahl M. et al. J. Am. Chem. Soc. 1991, 113:9366), by which the active site of an enzyme can be modified. Bio-imprinting is a method of modifying the specificity or selectivity of an enzyme for its substrate by modifying its active site. This is done by precipitation of the same enzyme in a solvent in the presence of the molecule of interest, which is not its natural substrate. The bio-imprinting method was applied for example by precipitation with 1-propanol of -chymotrypsin in the presence of N-acetyl-D-tryptophan, or N-acetyl-D-phenylalanine, or N-acetyl-D-tyrosine, followed by drying the precipitate. This bio-imprinting process has been shown to induce a new conformation of the active site, such that the active site after bio-imprinting binds the non-natural substrate provided.

In view of the aforementioned state of the art, the Applicants have set themselves the primary objective of providing a molecularly imprinted polymer in nanoparticle form, and a preparation method thereof, which has high biocompatibility and can thus be advantageously used for a variety of applications, for example in the fields of medicine, bioengineering and molecular sensing.

The Applicants have found that this and other purposes, which will be better explained in the following description, can be achieved by using a molecularly imprinted polymer prepared by crosslinking certain functionalized polymers in place of the conventional monomers used in the state of the art.

Therefore, according to a first aspect, the present invention relates to polymeric nanoparticles having recognition sites of at least one target molecule according to claim 1.

In accordance with a second aspect, the present invention relates to a cell decorated with the above-mentioned polymeric nanoparticles according to claim 12. In accordance with a third aspect, the present invention relates to a method for preparing the aforementioned polymeric nanoparticles according to claim 14.

In accordance with a fourth aspect, the present invention relates to the use of the above-mentioned polymeric nanoparticles according to claim 16.

In accordance with a further aspect, the present invention relates to a molecularly imprinted polymer in the form of nanoparticles according to claim 17. Further characteristics are identified in the dependent claims.

The present invention thus relates to the formation of selective biocompatible and biodegradable nanomaterials formed around a template molecule that is the target molecule of interest, a portion thereof, or a chimera thereof. Such nanomaterials are natural or hybrid polymers, mixed natural and synthetic, at least one of which is natural, wherein the steric configuration formed in the interaction between the template molecule and the polymer(s) is stabilized by crosslinking.

In a fundamental departure from previous strategies for forming specific binding sites using the molecular imprinting method, the present invention initiates the imprinting process from a preformed polymer, rather than from polymerizable monomers.

In the present invention, a polymer, whether it is a polyamide or a polysaccharide, but not limited thereto, or a mixture of said polymers, constitutes the element (building element) with which the imprinting process is carried out. The said polymer, or polymeric mixture, is partially or fully solvated and functionalized with reactive pendants (i.e., reactive groups), which include but are not limited to reactive double bonds (i.e. crosslinkable double bonds), fluorophores, reporter tags, chelating agents, hydrophilic groups, hydrophobic groups, charged groups. The aforementioned polymer, or polymeric mixture, is then placed in the presence of the molecule to be imprinted. Thermodynamics drive the interaction between the functionalized polymer chains and the template molecule. The three-dimensional rearrangement of the functionalized polymer chains around the template molecule is stabilized by chemical or photo-induced polymerization. The material thus prepared is characterized by its nanometric size and specific, selective binding sites for the template molecule. The modulation and optimization of the selectivity and specificity of said nanomaterial towards the template molecule is achieved by appropriately choosing the polymer or polymeric mixture to be used as a building element.

The method described enables the preparation of selective biocompatible nanomaterials with additional functionalities, including but not limited to fluorescent tags, polar, non-polar, chelating agents, charged functions. These additional functionalities are used in applications of the materials covered by the invention.

For the purposes of the present invention, the term “biocompatible” means that the element to which this term refers can come into contact with the human or animal body (e.g. biological fluids, living cells and tissues) without producing an undesirable response, such as an inflammatory, irritative or immune response.

The biocompatible nanomaterial according to the present invention is used in the selective recognition of analytes, in assays, for in vivo and in vitro targeting uses, labelling of specific biological molecules, including proteins and epitopes, delivery of drugs and active pharmacological ingredients to tissues, cells, in scaffolds for tissue growth and tissue repair. The biocompatible nanomaterial is also used in the preparation of degradable sensor elements suitable for the circular economy and for the preparation of environmentally friendly devices.

In a further embodiment, the present invention relates to biocompatible nanoparticles with specific selective binding sites formed by photo-induced or chemical crosslinking of at least one polymer modified with reactive groups and solvated in aqueous fluids, or in biocompatible polar solvents, or in biocompatible non-polar solvents, in the presence of the template molecule and with the addition of a catalyst.

In a further embodiment, the present invention relates to biocompatible nanomaterials, wherein the molecule to be imprinted is solvated, suspended in solution, or immobilized on a solid phase support.

In a further embodiment, the present invention relates to biocompatible nanomaterials, wherein the molecules to be imprinted are small molecules, peptides, proteins, enzymes, supramolecular complexes, cell portions, cells, bacteria, viruses.

In a further embodiment, the present invention relates to biocompatible nanomaterials, wherein the polymers are of natural origin, such as polyamides (including, but not limited to: collagen, gelatin, silk fibroin, sericin, fibrinogen, fibrin, elastin) and polysaccharides (including, but not limited to: chitin, keratin, chitosan, alginate, hyaluronic acid, starch, cellulose) or combinations or fractions thereof.

In a further embodiment, the present invention relates to biocompatible nanomaterials in which natural polymers are blended with synthetic polymers including, but not limited to: polyethylene glycol, polyvinyl alcohol, polyhydroxyethyl methacrylate, poly--caprolactone, polylactic acid, polyglycolic acid and derivatives thereof.

In a further embodiment, the present invention relates to biocompatible nanomaterials, wherein the diameter of the nanoparticles is in the range 10-1000 nm and preferably in the range 10-300 nm.

In a further embodiment, the present invention relates to biocompatible nanomaterials, wherein the size of the nanoparticles is adjusted by modulating the pH of the solvating fluid, when—but not only—the solvating fluid is water.

In a further embodiment, the present invention concerns biocompatible nanomaterials used as specific separation and enrichment materials.

In a further embodiment, the present invention relates to biocompatible nanomaterials used as recognition elements for producing degradable sensors, also starting from industrial waste and by-products of manufacturing processes, aimed at the circular economy.

In a further embodiment, the present invention relates to biocompatible nanomaterials used for targeting cells, pathogens and viruses, including in vivo applications.

In accordance with a further aspect, it is an object of the present invention to provide a molecularly imprinted polymer in nanoparticle form which is prepared using at least one peptide or polypeptide as the sole building element or as the predominant building element of the set of building elements used to form the polymer. That is, the set of building elements contains peptides and/or polypeptides in a preponderant amount with respect to building elements other than peptides and polypeptides. Therefore, according to the present invention, the set of building elements comprises a total number of moles of peptides and polypeptides higher than the total number of moles of building elements having a structure other than peptides and polypeptides. According to the invention, therefore, a substantial part of the polymeric structure of the nanoparticle is formed by the aforementioned crosslinked peptides and/or polypeptides.

It has been observed that the use of peptides and polypeptides as the only building element or as the predominant building element for the preparation of a molecularly imprinted polymer in the form of nanoparticles enables a highly biocompatible nanomaterial to be obtained, thus overcoming the drawbacks of nanomaterials of the prior art.

Furthermore, since peptides and polypeptides have a multitude of different functional groups (e.g., the R side chains of amino acids), they can interact with a wider variety of template molecules, and thus targets, than functional monomers of the prior art, such as (meth)acrylates, (meth)acrylamides and polysaccharides, thus leading to the synthesis of a correspondingly greater variety of molecular imprinted polymers.

In addition, the molecularly imprinted polymer in nanoparticle form can be prepared in a huge variety of structural architectures due to the many varieties of two- and three-dimensional configurations that characterize protein molecules (e.g. alpha-helices, beta structures). The molecular imprinted polymer is also characterized by high structural stability resulting from the large number of covalent and non-covalent bonds that can be formed between spatially adjacent building elements.

Therefore, according to a further embodiment, the present invention relates to a molecularly imprinted polymer in the form of a nanoparticle having recognition sites of at least a portion of a target molecule, wherein said nanoparticle comprises a three-dimensional polymeric structure formed by a set of crosslinked building elements, and wherein said set comprises:
a first fraction of building elements consisting of at least one peptide and/or polypeptide functionalized with at least one first crosslinkable group;
optionally, a second fraction of building elements consisting of at least one product having a structure other than a peptide or polypeptide, said product being functionalized with at least one second crosslinkable group;

wherein said peptides and/or polypeptides are present in an amount higher than 10 mol % in relation to the total moles of said building elements of said first and second fraction.

In a further embodiment, the present invention relates to a method for preparing the aforementioned molecularly imprinted polymer in nanoparticle form comprising the following steps in sequence:
a. preparing a polymerizable mixture comprising:
a1. at least one solvent,
a2. at least one set of building elements comprising:
   a first fraction of building elements consisting of at least one peptide and/or polypeptide functionalized with at least one first crosslinkable group,
   optionally, a second fraction of building elements consisting of at least one product having a structure other than a peptide or polypeptide, said product being functionalized with at least one second crosslinkable group,

wherein said peptides and/or polypeptides are present in an amount higher than 10 mol % in relation to the total moles of said building elements of said first and second fraction;
a3. at least one template molecule (or templating agent),
a4. at least one polymerization initiator;
b. polymerizing the polymerizable mixture to obtain a nanoparticle comprising a three-dimensional polymeric structure formed by said crosslinked first and second fractions and containing said template molecule;
c. removing said template molecule to obtain said molecularly imprinted polymer in the form of a nanoparticle having recognition sites of at least a portion of a target molecule.

The nanoparticles consist of a plurality (set) of building elements crosslinked together around the template molecule to form a three-dimensional polymeric structure (network) which, after removal of the template molecule, comprises recognition sites having a shape complementary to said molecule.

The building elements forming the three-dimensional polymer structure comprise at least one crosslinkable functional group, e.g. a crosslinkable double bond.

The set of building elements comprises a first fraction of building elements consisting of peptides and/or polypeptides, each being functionalized with at least one first crosslinkable group.

Optionally, the set of building elements comprises a second fraction of building elements comprising products that are not peptides or polypeptides, for example (meth)acrylate or (meth)acrylamide monomers, each being functionalized with at least one second crosslinkable group. The aforementioned first and second crosslinkable groups may be the same or different from each other.

In one embodiment, the set of building elements comprises only the first fraction of building elements consisting of peptides, polypeptides or a combination thereof.

The set of building elements can comprise, for example, up to 30 different types of building elements. Preferably the total number of types of building elements present in the first and second fraction is in the range 1-10, more preferably in the range 1-6.

Preferably, the number of different building elements present in each of the first and second fractions is selected, independently, in the range 1-5, more preferably in the range 1-3.

The peptides and/or polypeptides are present in the set of building elements in a total amount greater than 10 mol % relative to the total moles of the building elements of the first and second fraction of building elements of the set, more preferably greater than or equal to 25 mol %, even more preferably greater than or equal to 50 mol %, preferably greater than or equal to 65 mol %, more preferably greater than or equal to 75 mol %, even more preferably greater than or equal to 85 mol %, even more preferably in the range 90-100 mol %.

For the purposes of this description, a peptide is a linear chain containing from 2 to 20 amino acid residues.

Preferably, the peptide has a molecular weight in the range 200-2000 Daltons.

For the purposes of this description, the term ‘polypeptide’ means a polymer of amino acids of any length and molecular weight. Polypeptides that can be used in the present invention also include proteins.

Preferably, the polypeptide has a molecular weight in the range 2,100-1,000,000 Daltons, preferably 10,000-300,000 Daltons.

Preferably, the polypeptide is selected from: silk fibroin, collagen, gelatin, fibrin, elastin, beta-structured protein, laminin, fibronectin, keratin, albumin, sericin, fibrinogen and combinations thereof.

In one embodiment, the peptide or polypeptide is a recombinant peptide or polypeptide.

In a particularly preferred embodiment, the polypeptide consists of silk fibroin.

The second fraction of building elements consists of at least one product having a structure other than a peptide or polypeptide, which is functionalized with at least one second crosslinkable group. Examples of functionalized products of the second fraction include: biodegradable polymers and monomers thereof capable of forming a biodegradable polymer, biocompatible polymers and monomers thereof capable of forming a biocompatible polymer, or (meth)acrylates, (meth)acrylamides, (meth)acrylic acid and the like.

Further examples of functionalized products of the second fraction include: chitin, chitosan, alginate, hyaluronic acid, starch, cellulose, polyamides, polysaccharides and combinations thereof.

Examples of biocompatible and/or biodegradable polymers are: poly(meth)acrylic acid, poly(2-hydroxyethyl methacrylate), polyamide, polyacrylamide, polyethylene glycol, polyvinyl alcohol, polylactic acid, polyglycolic acid, polyurethane and copolymers or mixtures thereof.

The crosslinkable functional groups of the building elements of the first and second fraction can be selected from a wide variety of groups. Examples of crosslinkable groups include: (meth)acrylate, (meth)acrylamide, vinyl ether, epoxide, diene, thiol, alcohol, amine, carboxylic acid, succinimide, glycidyl ether, siloxane, alkane and azide.

Preferably, the at least one crosslinkable group comprises or consists of a double bond.

Preferably, the at least one crosslinkable group is an acryloyl or methacryloyl group resulting from the conjugation of acrylate and methacrylate compounds with the protein molecule.

The number of crosslinkable groups on the building elements can vary, for example, from 1 to 10 for low molecular weight building elements to several hundred for polymeric building elements.

Crosslinkable functional groups can be introduced into the building element of the first and second fraction by methods known to a person skilled in the art. For example, peptides and polypeptides can be functionalized by means of a (meth)acrylation reaction. Alternatively, compounds containing a crosslinkable group (e.g. acryloyl) functionalized with N-hydroxysuccinimide (NHS) can be reacted with the lysine residues of the peptide or polypeptide.

Alternatively, compounds containing two N-hydroxysuccinimides (NHS) at the extremities can be reacted with the lysine residues of the peptide or polypeptide.

In one embodiment, peptides and polypeptides may also comprise additional functional groups capable of performing specific functions, such as fluorophore, reporter tag, chelating, hydrophilic, hydrophobic, electrically charged groups and combinations thereof.

It is also possible to introduce functional groups into the molecularly imprinted polymer by incorporating specific crosslinkable building elements into the set of building elements. For example, it is possible to obtain polymeric nanoparticles containing fluorescent tags, using crosslinkable compounds with fluorescent groups, such as: methacryloxyethyl thiocarbamoyl rhodamine B; 9-anthracenylmethyl acrylate or fluorescein o-acrylate.

A templating agent or template molecule is used to imprint the recognition site in the polymer. As the templating agent, either the entire target molecule for which a polymer with binding affinity is desired or a portion of said target molecule can be used.

For the purposes of this description, the terms “target molecule” and “target” are used interchangeably and identify the molecule, molecular structure or portions thereof for which the polymer possesses recognition sites.

The target can vary in size over a wide range of values. The target may be, for example, a small molecule or a biological macromolecule, e.g. a protein, or a complex biological structure, such as a virus or a portion of a virus, the surface of a cell or a portion of said surface. The target may also be an inorganic structure. The target may be a natural or synthetic molecule or structure.

In one embodiment, the target is chosen from: peptide, protein, enzyme, supramolecular complex, cell or portion thereof, bacterium, virus, pharmacological agent, biological modifier, diagnostic agent and combinations thereof.

The molecularly imprinted polymer according to the present invention is in the form of nanoparticles having a diameter of up to about 1,000 nm, preferably from 10 nm to 1,000 nm, more preferably from 10 nm to 300 nm. The size of nanoparticles can be determined e.g. by dynamic light scattering (DLS) measurements.

The molecularly imprinted polymer in nanoparticle form can be prepared in accordance with techniques known to a person skilled in the art. It may be obtained, for example, by polymerizing, in the presence of at least one polymerization initiator, a polymerizable mixture comprising at least one solvent, at least one set of building elements, at least one templating agent, and subsequently removing the template from the polymer matrix formed.

The polymerizable mixture may be prepared by mixing in the solvent the peptides and/or polypeptides of the first fraction with the building elements of the second fraction, if present, together with the template molecule, the polymerization initiator and any additional optional components.

The solvent is preferably a polar solvent or a non-polar solvent, preferably biocompatible.

Examples of polar solvents include: water: N-methyl-2-pyrrolidone, 2-pyrrolidone, polyethylene glycol, dimethyl sulfoxide, ethyl lactate, dimethylacetamide.

Examples of non-polar solvents include: triacetin, ethyl acetate, propylene carbonate, triethyl citrate, ethyl heptanoate, benzyl alcohol, benzyl benzoate.

In one embodiment, the solvent is preferably water or, more preferably, a buffer solution capable of maintaining the pH at a desired value, e.g. in the pH range=3-9. It was surprisingly observed that, when the solvent is an aqueous solvent, the choice of pH value in this range determines the average size of the nanoparticles and the polydispersion index of the polymer.

In general, in order to obtain the molecularly imprinted polymer according to the invention, the polymerization reaction may be carried out by one of the polymerization methods known to a person skilled in the art, such as, for example: high dilution polymerization, precipitation polymerization, suspension polymerization, emulsion polymerization and dispersion polymerization.

Preferably, polymerization is conducted under conditions of high dilution of the set of building units. In one embodiment of the high dilution polymerization, the concentration of the set of building units in the polymerization mixture is in the range 0.005%-2% w/v, preferably in the range 0.01%-1% w/v.

The polymerization reaction to make the polymer matrix of the molecular imprinted polymer is preferably a radical polymerization reaction. Preferably, the polymerization is varied out by photo-crosslinking or chemical crosslinking. In a particularly preferred embodiment, the polymerization is a UV photopolymerization carried out in the presence of a radical photoinitiator.

The polymerization initiator can therefore be chosen, for example, from the initiators used in UV radiation polymerization. The photo-crosslinking initiator, for example, may be chosen from: lithium phenyl-2,4,6-trimethyl-benzoyl phosphinate, azobis(2-methylpropionamidine) dihydrochloride, and mixtures thereof.

Alternatively, the polymerization can be carried out in the presence of a thermal polymerization or Red-Ox initiator.

Preferably, the polymerization initiator is present in the polymerizable mixture at a concentration in the range of 0.005%-0.5% w/v, more preferably in the range of 0.01%-0.5% w/v.

The templating agent may be dissolved or suspended in the polymerization mixture or immobilized on a solid support, according to techniques known to a person skilled in the art.

The concentration of the templating agent in the polymerization mixture can vary over a wide range depending on the composition of the templating agent and the protein molecule, and can be easily determined by a person skilled in the art according to their knowledge.

The polymerizable mixture may optionally contain a crosslinking agent, i.e. a molecule having at least two functional groups that can polymerize with the building units of the polymerizable mixture. Crosslinking agents known in the art may be used for this purpose. Examples of crosslinking agents that can be used are: N,N′-methylenebis-acrylamide, polyethylene glycol dimethacrylate, divinylbenezene, 3-(acryloyloxy)2-hydroxypropyl methacrylate.

In one embodiment, the overall concentration of crosslinker in the monomer mixture is in the range of 0.01-5% w/v.

According to a further aspect, the present invention thus relates to the polymerizable mixture described above.

Preferably, the polymerization reaction is conducted at a temperature in the range 10-40° C., more preferably in the range 20-35° C.

At the end of the polymerization reaction, the molecularly imprinted polymer is in the form of nanoparticles suspended in the solvent. The polymer is then treated to remove the templating agent from the polymer matrix and create recognition sites for the target molecule. The removal of the templating agent can be achieved, for example, by dialysis or ultrafiltration. If the templating agent is a protein, this can be removed using a solution containing an enzyme, e.g. trypsin.

The suspension comprising the polymeric nanoparticles can be used as such, e.g. to prepare a final product comprising the nanoparticles, such as complex macromolecular structures, molecular sensors, medical devices for in vivo applications (e.g. patches) or for devices in the field of tissue engineering and regenerative medicine (TERM).

According to a further aspect, the present invention thus relates to a liquid suspension, preferably an aqueous suspension, comprising the molecular imprinted polymer in nanoparticle form.

Alternatively, the particles can be recovered in solid form from the suspension by freeze-drying.

The molecular imprinted polymer in nanoparticle form can be used for numerous applications in medicine, bioengineering, molecular sensors, optics, opto-electronics and, in general, for all applications where the use of molecularly imprinted polymers in nanoparticle form is envisaged.

In particular, the molecularly imprinted polymer in nanoparticle form is used in the selective recognition of analytes, in assays, for in vivo and in vitro targeting, labelling of specific biological molecules, including proteins and epitopes, administration of drugs and active pharmacological ingredients to tissues (e.g. as carriers of active pharmacological ingredients), cells, in scaffolds for tissue growth and tissue repair. The molecularly imprinted polymer according to the present invention is also used in the preparation of sensor elements, in particular degradable sensors suitable for the circular economy, and environmentally friendly devices.

In particular, according to a further aspect, the present invention relates to a decorated cell or fibre, for example for biomedical use, by means of the molecular imprinted polymer in nanoparticle form.

The fibre can be natural or synthetic, for example the type used as a scaffold in TERM applications. In one embodiment, the fibre comprises silk fibroin. Fibre decoration can be carried out in accordance with known techniques, for example by means of crosslinking compounds such as carbodiimides or succinimides.

The molecularly imprinted polymer according to the present invention in nanoparticle form can also be used for the separation and specific enrichment of target molecules in mixtures with other components and for the targeting of cells, pathogens and viruses.

The following examples are intended to illustrate the invention, but are not limited thereto.

In the following examples, reference will be made to the accompanying figures, which illustrate:

FIG. 1, Size distribution of nanoparticles of selective biocompatible nanomaterials according to the invention determined by dynamic light scattering measurements;

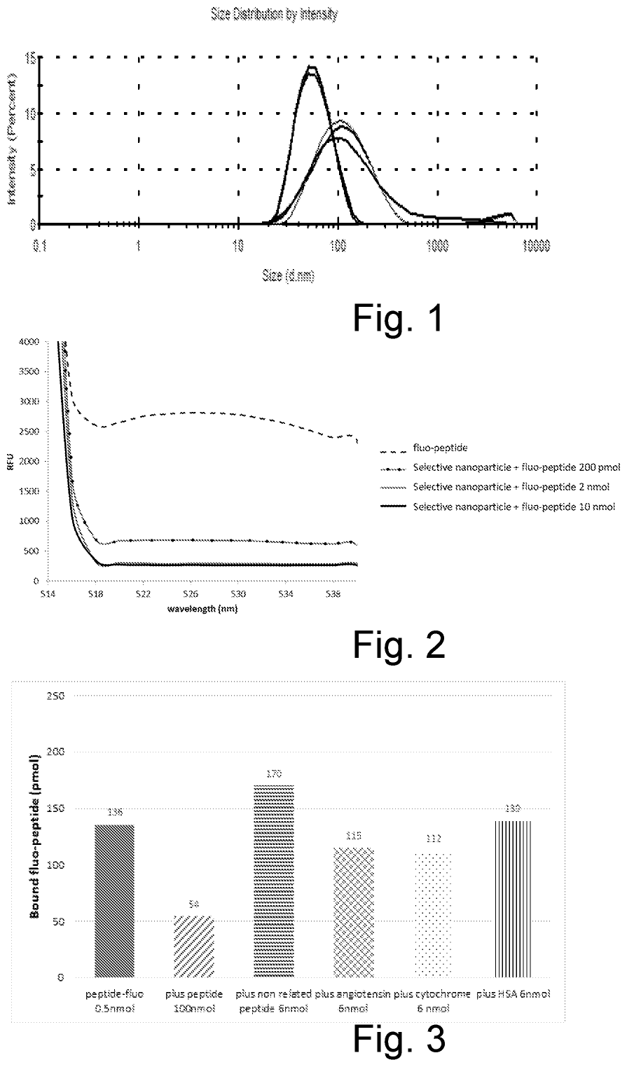

FIG. 2, Fluorescent signal of selective biocompatible nanomaterials of free target molecules and bound to the imprinted cavity;

FIG. 3, Proof of selectivity of nanomaterials according to the invention;

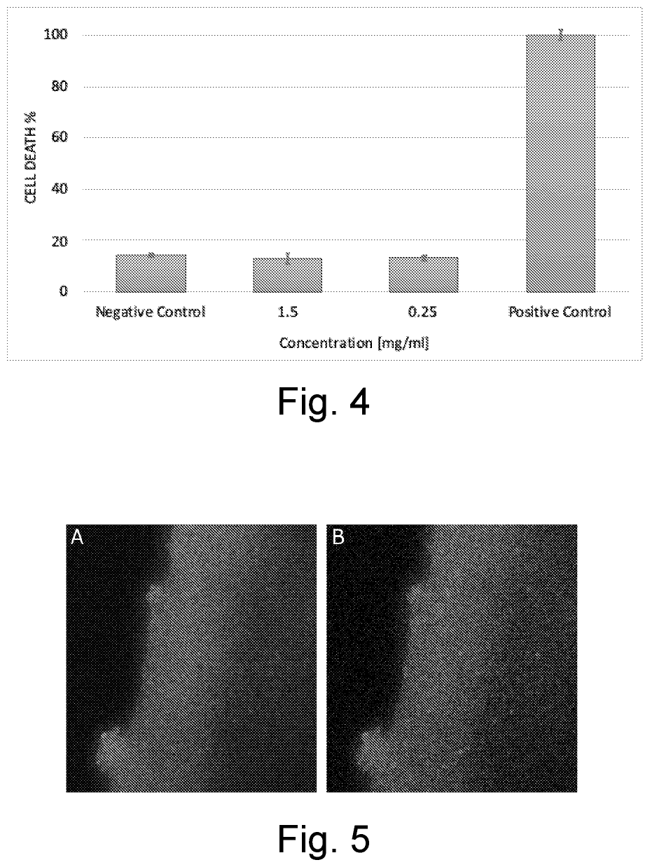

FIG. 4, Proof of non-toxicity of nanomaterials according to the invention;

FIG. 5, Fluorescence images of 3T3 cells decorated with nanomaterials according to the invention: (A) cytoskeleton; (B) rhodamine-tagged nanomaterials.

### Example 1. SELECTIVE FIBROIN-BASED NANOMATERIALS

By way of example, molecularly imprinted silk fibroin nanoparticles are produced, i.e. a molecular imprinted polymer obtained using silk fibroin as the sole building block. Silk fibroin offers interesting characteristics, such as its natural origin, which gives it biocompatibility, and makes it suitable for numerous uses, including but not limited to tissue engineering, in vivo and in vitro cell targeting, drug delivery (Altman G G. et al. Biomaterials 2003, 24:401, Nazarov R. et al. Biomacromolecules 2004, 5: 718, Mottaghitalab F. et al. J. Control. Release 2015, 206:161). Silk fibroin has special mechanical properties (Lawrence B D. et al. J. Mater. Sci. 2008, 4:6967; Sofia S. et al. J. Biomed. Mater. Res. 2001, 54:139; Meinel L. et al. Bone 2006, 39: 922; Jin H.-J. et al. Biomacromol. 2002, 3:1233), biological properties (Santin M. et al. J. Biomed. Mater. Res. 1999, 46:382; Pritchard E. M. et al. J.Control. Release 2010, 144:159) and optical properties (Perry H. et al. Adv. Mater. 2008, 20:3070; Lawrence B. D. et al. Biomacromol. 2008, 9:1214) indicated for use in the biomedical, optical, opto-electronic field (Perotto G. et al. Appl. Phys. Lett. 2017, 111:103702; Bay H. H. et al. Nano Lett. 2019, 19:2620; US2015368417A1).

To obtain silk fibre, Bombyx mori silk cocoons are cut into small pieces and placed in a high-temperature thermostatic bath in the presence of 0.01 M sodium carbonate (Na₂CO₃) for 1 hour. This is followed by a second bath in sodium carbonate at a concentration of 0.003 M for 1 hour. The resulting silk fibre is thoroughly rinsed three times using ultra-pure water and then dried for 2 days.

The resulting fibroin was then functionalized by methacrylation. For this purpose, 20 g of scoured silk fibre is dispersed in 100 mL of a 9.3 M aqueous solution of lithium bromide (LiBr) at 60° C. for 4 hours in an oven. Next, 10 mL of glycidyl methacrylate (GMA) is added to the suspension, which is then shaken at 65° C. for 4 hours to allow the conjugation reaction to take place. To remove the salt and unreacted GMA, the resulting methacrylate fibroin suspension is dialyzed for 4 days against water using a dialysis system with a molecular cut-off corresponding to 3.5 kDa, filtered through a 50 μm glass filter and then stored at 4° C. until use.

The nanoparticles are obtained by high-dilution polymerization. In order to obtain the nanometric fibroin suspension, the concentration of methacrylate fibroin was then adjusted in the range of 0.01% to 0.5% w/v in aqueous buffer, specifically to the value of 0.03% w/v and the value of 0.3% w/v, in the presence of the template molecule, whether it is a small molecule, a peptide, or a protein. In the example provided here the use of bovine serum albumin (BSA) is shown, added at a final concentration of 0.1 mg/mL. A photoinitiator (e.g. lithium phenyl-2,4,6-trimethyl benzoyl phosphinate) is then added at a final concentration of 0.2% or 0.02% w/v and the suspension is photo-polymerized under UV light for 10 minutes to allow crosslinking. A population of nanometric particles is formed suitable for recognizing the template molecule, or a part thereof, or a chimera thereof. At the end of the crosslinking process, the template molecule is removed from the formed nanoparticles by several washes (subsequent dialysis against 4×3 L aqueous solutions, or ultrafiltration on 100 KDa molecular sized membranes with 3 L of aqueous solution). If the imprinted molecule is a protein, it is removed by adding trypsin enzyme to the material for 1 hour at room temperature and pH 8.0, followed by acidification of the solution, while the removal of the template protein from the nanomaterial is confirmed by SDS-PAGE electrophoresis.

The size of the imprinted biocompatible nanomaterials, estimated by dynamic light scattering, is in the range of 30-200 nm, the most represented hydrodynamic sizes in the prepared nanoparticle populations being 50 nm and 100 nm (FIG. 1). In FIG. 1, the two curves with maximum intensity at approximately 50 nm refer to the fibroin suspension at a concentration of 0.03% w/v (measurements made on two aliquots of the same sample), while the three curves with maximum intensity at approximately 100 nm refer to the fibroin suspension at a concentration of 0.3% w/v (measurements made on three aliquots of the same sample).

The binding properties of the imprinted biocompatible nanomaterials are tested by incubating the nanomaterial (0.1 mg/mL) in the presence of the modified template molecule with a fluorescent tag to form a fluorescent peptide (0.2 and 2 nmol) and monitoring the fluorescent signal over time. During binding of the fluo-peptide to the imprinted cavity, a decrease in the fluorescent signal is observed, because the amount of free fluo-peptide in solution decreases, while the amount of fluo-peptide bound to the imprinted cavities increases (FIG. 2).

The selectivity of the imprinted biocompatible nanomaterials is demonstrated by a 30-minute incubation of the nanomaterials (0.1 mg/mL) in the presence of either fluo-peptide alone (500 pmol), or alternatively by incubating the same amount of nanomaterial in the presence of a mixture consisting of the fluo-peptide (500 pmol) and one of the following competitor molecules: the same peptide used as the template molecule, but not fluorescently tagged (100 nmol); a biopeptide with a sequence unrelated to the peptide used as the template molecule, angiotensin (6 nmol); a protein with a sequence unrelated to the template peptide, namely cytochrome (6 nmol); an extremely abundant serum protein with a sequence unrelated to the template peptide, namely human serum albumin (6 nmol). The results demonstrate the preferential binding of the selective biocompatible nanomaterial to the template molecule. Competition is only observed when the untagged template molecule (the peptide) is present (FIG. 3).

The non-cytotoxicity of the prepared biocompatible selective nanomaterial is tested according to ISO 10993, using the NIH 3T3 cell line expanded with the respective standard medium and evaluated at a confluence of approximately 70%. The percentage of cell death was evaluated by measuring the amount of lactate dehydrogenase released into the medium from cells fed with medium containing a concentration of 0.25 and 1.5 mg/ml of nanoparticle suspension, then compared with the negative control (untreated cells, as reference for non-cytotoxic material) and positive control (all dead cells, as reference for totally cytotoxic material) (FIG. 4).

### Example 2. Selective Fibroin-Based Nanomaterials Functionalized with Fluorescent Tags

The preparation of functionalized selective biocompatible nanomaterials occurs as in Example 1, but includes the addition of polymerizable fluorescent tags (such as, for example: methacryloxyethyl thiocarbamoyl rhodamine B; 9-anthracenylmethyl acrylate; fluorescein o-acrylate) as additional building elements, used in the concentration range of 0.0002% to 0.02% w/v with respect to the concentration of the fibroin suspension. These biocompatible fluorescent nanomaterials are used to decorate cells (FIG. 5).

1. Polymeric nanoparticles having recognition sites of a target molecule, said polymeric nanoparticles being obtained by crosslinking of a functionalized polymer at least with polymerizable double bonds, in a liquid and in the presence of a target molecule as a template molecule.

2. The polymeric nanoparticles according to claim 1, wherein the functionalized polymer comprises a biocompatible polymer optionally in a mixture with a synthetic polymer.

3. The polymeric nanoparticles according to claim 2, wherein the biocompatible polymer is selected from: polyamides, polysaccharides and combinations thereof.

4. The polymeric nanoparticles according to claim 1, wherein the functionalized polymer is a natural polyamide selected from: collagen, gelatin, silk fibroin, sericin, fibrinogen, fibrin, elastin, chitin, keratin and combinations thereof.

5. The polymeric nanoparticles according to claim 4, wherein said nanoparticles are obtained by crosslinking building elements formed by only natural functionalized polymers.

6. The polymeric nanoparticles according to claim 5, wherein said nanoparticles are obtained by crosslinking a single functionalized polymer which is silk fibroin.

7. The polymeric nanoparticles according to claim 2, wherein said synthetic polymer is selected from: polyethylene glycol, polyvinyl alcohol, polyhydroxyethyl methacrylate, poly-ϵ-caprolactone, polylactic acid, polyglycolic acid and copolymers thereof.

8. The polymeric nanoparticles according to claim 1, wherein said functionalized polymer is functionalized at least with polymerizable double bonds by a methacrylation reaction.

9. The polymeric nanoparticles according to claim 1, wherein said functionalized polymer with at least polymerizable double bonds comprises one or more functional groups selected from: fluorophore groups, reporter tags, chelating groups, hydrophilic groups, hydrophobic groups, electrically charged groups.

10. The polymeric nanoparticles according to claim 1, wherein said template molecule is at least one chosen from: peptides, proteins, enzymes, supramolecular complexes, cell portions, cells, bacteria, viruses and combinations thereof.

11. The polymeric nanoparticles according to claim 1, wherein said liquid is selected from: water, polar solvent, biocompatible polar solvent, non-polar solvent, biocompatible non-polar solvent.

12. A decorated cell comprising polymeric nanoparticles according to claim 1.

13. A method for preparing the polymeric nanoparticles according to claim 1, comprising:
a. crosslinking the functionalized polymer at least with the polymerizable double bonds, in a liquid and in the presence of the target molecule as the template molecule to obtain polymer nanoparticles of a crosslinked polymer containing said template molecule; and
b. removing said template molecule to obtain said polymeric nanoparticles having the recognition sites of said target molecule.

14. The method according to claim 13, wherein the crosslinking is carried out by photo-crosslinking or chemical crosslinking.

15. The method according to claim 13, wherein the size of the polymeric nanoparticles is adjusted by modulating the pH of the liquid.

16. The polymeric nanoparticles according to claim 1, which are suitable for at least one of:
selective recognition of analytes in assays;
in vivo or in vitro targeting of a biological molecule;
labelling of biological molecules;
administration of drugs and pharmacologically active ingredients to tissues or cells;
growth and repair of tissues;
as a recognition element in a sensor; or
separation and enrichment of mixtures comprising said target molecule.

17. A molecularly imprinted polymer in the form of a nanoparticle having recognition sites of at least a portion of a target molecule, wherein said nanoparticle comprises a three-dimensional polymer structure formed by a set of crosslinked building elements, and wherein said set comprises:
a first fraction of building elements consisting of a peptide and/or polypeptide functionalized with a first crosslinkable group;
optionally, a second fraction of building elements consisting of a product having a structure other than a peptide or polypeptide, said product being functionalized with a second crosslinkable group;
wherein said peptides and/or polypeptides are present in an amount higher than 10 mol % in relation to the total moles of said building elements of said first and second fraction.

18. The polymeric nanoparticles according to claim 1, wherein the functionalized polymer comprises a biocompatible polymer of natural origin in a mixture with a synthetic polymer.

19. The polymeric nanoparticles according to claim 4, wherein said nanoparticles are obtained by crosslinking building elements formed by only natural functionalized polymers selected from natural polyamides.

20. The polymeric nanoparticles according to claim 1, wherein said template molecule is bovine serum albumin.
